(19) United States
(12) Patent Application Publication
(10) Pub. No.: US 2006/0067994 A1
(43) Pub. Date: Mar. 30, 2006

(54) System for providing therapy to a body

(75) Inventor: Ricky W. Purcell, Alpharetta, GA (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(73) Assignee: Kimberly-Clark Worldwide, Inc.

(21) Appl. No.: 10/954,764

(22) Filed: Sep. 30, 2004

(51) Int. Cl. A61K 9/70

(52) U.S. Cl. 424/449

(57) ABSTRACT

Some embodiments of the present invention relate to a system, kit and method for providing therapy to a portion of a body. The system includes a holder and a therapeutic patch that is held by the holder. The therapeutic patch applies therapy to the portion of the body when the first patch is placed near the portion of the body. The system further includes an oral pain reliever that is held by the holder. The method includes selecting a therapeutic patch from a holder and applying the therapeutic patch near a portion of a body such that the therapeutic patch applies therapy to the portion of the body. The method further includes selecting an oral pain reliever from the holder and ingesting the oral pain reliever.

### FIELD

This invention relates in general to a system for providing therapy to a body, and in particular to a system that readily provides therapy using a therapeutic patch in combination with an oral pain reliever.

### BACKGROUND

Applying therapy to various parts of a body is a recognized practice for curing or alleviating multiple kinds of physical problems. One example therapy involves cooling an injured portion of a body by applying ice packs near the injured portion of the body in an effort to reduce swelling, inflammation and/or muscle pain.

Another cooling method utilizes a cold pack that generates cooling via an endothermic chemical reaction which takes place within the cold pack. The cold pack typically includes an endothermic solute and a liquid that are both stored within a common enclosure. The liquid and solute are initially segregated from another within the enclosure and then mixed within the enclosure to form an endothermic solution that reduces the temperature of the cold pack.

Another example therapy utilizes heat to treat symptoms such as stiffness, muscle pain, cold hands and feet, lumbago, rheumatism and neuralgia (among others). Some known heat-treating methods include direct application of heat to the body using items such as a towel, jelly and/or paste. Another example heating therapy includes applying a heating pad to an injured portion of a body to alleviate discomfort.

Some heat-treating methods utilize heat patches to apply heat to a body. One such heat patch generates heat via an exothermic chemical reaction that takes place within the heat patch. Heat patches that generate heat using an exothermic reaction typically include an enclosure and a heating composition stored within the enclosure. At least a portion of the enclosure is air-permeable such that exposing the heating composition within the heat patch to air generates a heat-producing exothermic reaction.

Another type of therapy utilizes infrared energy reflectors to reflect infrared energy emitted by the body back into an injured portion of the body. The infrared energy reflectors are sometimes part of a patch that includes a reflective layer.

Electrical stimulation may also be used to apply therapy to an injured portion of a body. The electrical stimulation is typically applied by a device that is positioned on or near the injured portion of the body. Some of the electrical devices that are used to apply electrical stimulation include their own power supply while others must be connected to an external power supply.

Another type of therapy utilizes counter irritants (e.g., menthol, camphor) to mask pain at an injured portion of the body. The counter-irritants are sometimes part of a patch that is applied to the injured portion of the body.

Another form of therapy includes orally ingesting a pain reliever to treat pains in muscles, joints and many other areas of the body. One drawback with oral pain relievers is that they delivered indiscriminately through the blood stream over the entire body instead of being directed to an injured area of the body.

### SUMMARY OF THE INVENTION

The present invention relates to a system and method for providing therapy to a portion of a body. The system and method allow a user or therapist to readily apply a therapeutic patch to an injured area of the body while more efficiently directing an oral pain reliever to the injured area of the body. The system and method also reduce the need to purchase and store excessive inventories of therapy-related products.

In one aspect, the system includes a holder and a therapeutic patch that is held by the holder. The therapeutic patch applies therapy to the injured portion of the body when the therapeutic patch is placed near the portion of the body. The system further includes an oral pain reliever that is held by the holder. In some embodiments, the therapeutic patch within the system may generate a physiological response at the injured portion of the body such that the oral pain reliever is more efficiently directed to the injured area of the body.

In another aspect, the present invention relates to a kit of parts for providing therapy to a portion of a body. The kit includes a therapeutic patch that is adapted to apply therapy to the portion of the body when the therapeutic patch is placed near the portion of the body. The kit further includes an oral pain reliever that is adapted to relieve pain from the body when the oral pain reliever is ingested within the body and a holder that is adapted to hold the therapeutic patch and the oral pain reliever.

The kit may provide a user or therapist with the ability to readily provide an effective combination of therapies to an injured area of a body. In addition, the kit reduces the need to stock (i.e., inventory) the multiple containers, boxes, etc. that would otherwise normally be necessary to provide a combination of therapies.

In yet another aspect, the present invention relates to a method of providing therapy to a portion of a body. The method includes selecting a therapeutic patch from a holder and applying the therapeutic patch near the portion of the body such that the therapeutic patch applies therapy to the portion of the body. The method further includes selecting an oral pain reliever from the holder and ingesting the oral pain reliever.

The purposes and features of the present invention will be set forth in the description that follows. Additional features of the invention will be realized and attained by the product and processes particularly pointed out in the written description and claims hereof, as well as from the appended drawings.

It is to be understood that both the foregoing general description and the following detailed description are exemplary and are intended to provide further explanation of the invention claimed. The accompanying drawings, which are incorporated in and constitute part of this specification, are included to illustrate and provide a further understanding of the invention.

### BRIEF DESCRIPTION OF THE DRAWINGS

The present invention will be more fully understood, and further features will become apparent, when reference is made to the following detailed description and the accompanying drawings. The drawings are merely representative and are not intended to limit the scope of the claims. It should be noted that like parts which are depicted in the drawings are referred to by the same reference numerals.

FIG. 1 illustrates a perspective view of a system for providing therapy to a portion of a body.

FIG. 2 illustrates a perspective view of a therapeutic patch from the system of FIG. 1 where the therapeutic patch is mounted near an injured portion of a body.

FIG. 3 illustrates a perspective view of a plurality of therapeutic patches joined together.

FIG. 4 illustrates a perspective view of one of the therapeutic patches shown in FIG. 3 where the therapeutic patch is partially removed an enclosure.

FIG. 5 shows an example flexible wrap that may be used in some systems of the present invention where a therapeutic patch forms part of the flexible wrap.

FIG. 6 shows an example oral pain reliever that may be used in some systems of the present invention where the oral pain reliever is sealed inside an enclosure.

FIG. 7 illustrates a perspective view of another system for providing therapy to a portion of a body.

FIG. 8 illustrates a perspective view of a kit for providing therapy to a portion of a body.

### DETAILED DESCRIPTION OF THE INVENTION

In the following detailed description, reference is made to the accompanying drawings, which show specific embodiments in which the invention may be practiced. These embodiments are described in sufficient detail to enable those skilled in the art to practice the invention. It is to be understood that other embodiments may be utilized and structural changes made, such that the following detailed description is not to be taken in a limiting sense.

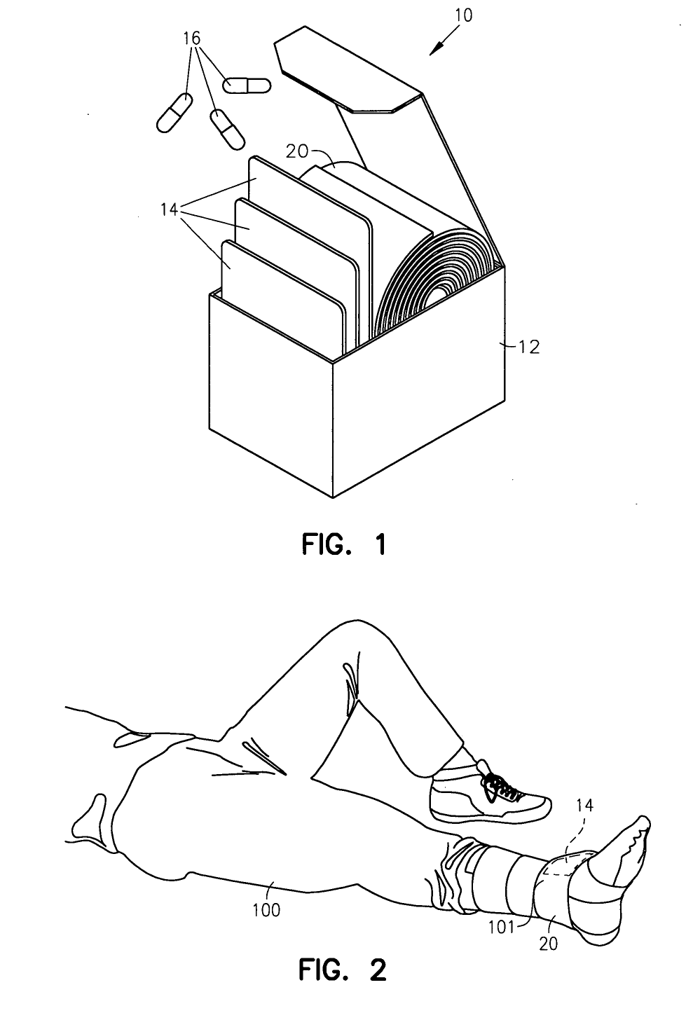

FIG. 1 illustrates a system 10 for providing therapy to a portion of a body. The system 10 includes a holder, such as container 12, and one or more therapeutic patches 14 that are held by the container 12. As shown in FIG. 2, the therapeutic patch 14 applies therapy to the portion 101 of the body 100 when the therapeutic patch 14 is placed on, or near, the portion 101 of the body 100.

The system 10 further includes one or more oral pain relievers 16 that are held by the container 12. The oral pain reliever 16 applies therapy to the portion 101 of the body 100 when the oral pain reliever 16 is ingested within the body 100.

Using a combination of a therapeutic patch 14 and an oral pain reliever 16 to apply therapy may either increase the effectiveness of a given dosage of oral pain reliever 16, or match a given effectiveness at a lower dosage. As discussed above, one drawback with orally ingested pain relievers is that they are delivered indiscriminately throughout the body via the bloodstream. Using the therapeutic patch 14 to generate a physiological change near the injured area 101 of the body 100 may positively impact the amount of oral pain reliever 16 that is delivered to the injured area 101 of the body 100 through the blood stream.

As an example, the temperature of an injured area of the body may be elevated by the therapeutic patch 14 as an oral pain reliever 16 (e.g., a Non-Steroidal Anti-Inflammatory Drug (NSAID)) is ingested. Elevating the temperature of the injured area 101 may generate vasodilatation within the injured area 101 thereby increasing the amount of blood within the injured area 101. Increasing the amount of blood within the injured area would increase the amount of oral pain reliever within the injured area at a given point in time

Elevating the temperature of the injured area 101 may also (i) increase the blood flow to the injured area 101; and/or (ii) increase the capillary permeability within the injured area 101. Increasing the blood flow to the injured area 101 would also increase the rate at which the oral pain reliever is delivered to the injured area. In addition, increasing the capillary permeability may allow a greater percentage of the oral pain reliever 16 to be delivered from the blood stream to the injured area 101 of the body 100 as compared to uninjured areas.

Therefore, elevating the temperature of the injured area may result in an increased concentration of the oral pain reliever 16 within the injured area 101 such that there is increased efficacy for a given dosage. In addition, elevating the temperature of the injured area 101 may allow the same efficacy to be achieved at a smaller dosage thereby reducing the impact of adverse side effects that are associated with ingesting the oral pain reliever 16.

Although FIG. 1 illustrates a box being used as container 12, it should be noted that any type of container may used in the system 10. Some other example containers include cans, jars and pouches (among others).

In the sample system 10 illustrated in FIG. 1, a plurality of therapeutic patches 14 are held by the container 12. Although three patches 14 are shown in FIG. 1, the number and type of patches may vary depending on the type of system. As an example, one therapeutic patch 14 may be an exothermic heating patch and another therapeutic patch 14 may an endothermic cooling patch. Another example therapeutic patch 14 may include a counter-irritant that masks pain when the therapeutic patch 14 is applied to the portion 101 of the body 100.

It should be noted that any of the therapeutic patches 14 described herein may include an infrared energy reflector (e.g., an aluminized film) and an adhesive area that may be used to attach the therapeutic patch 12 to the body 100. Other example therapeutic patches include electrical stimulating devices and devices that deliver acupressure therapy (among others). As used herein, therapeutic patch is meant to refer to packs, packages, inserts, buttons and/or any other device that is readily applied near an injured portion of a body to apply therapy. The choice of which therapeutic patch 14 to apply will depend on the type of therapy that is required by the injured portion 101 of the body 100.

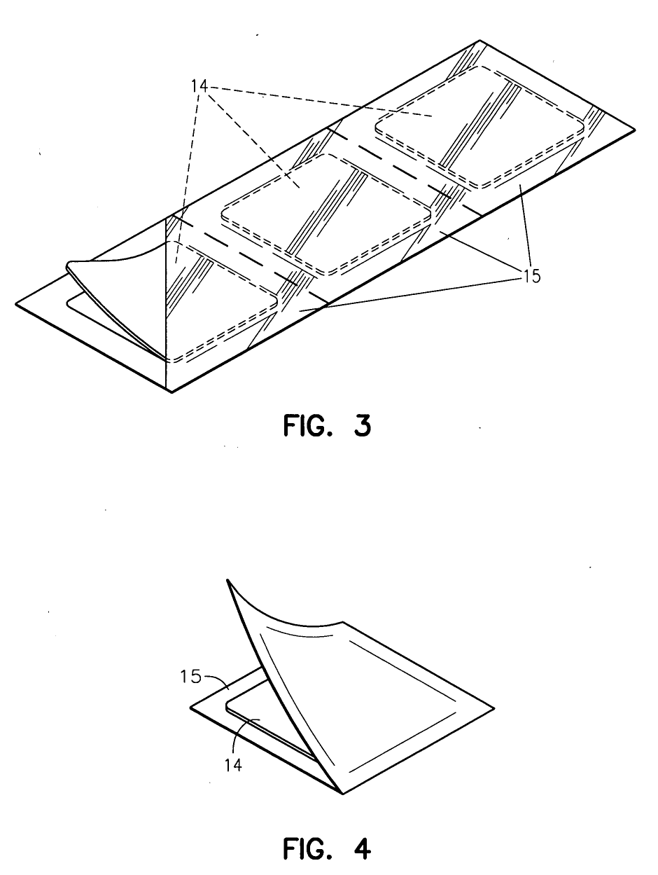

As shown in FIG. 3, a plurality of therapeutic patches 14 may be joined together such that the plurality of therapeutic patches 14 can be readily stored in the container 12. The system 10 may include a plurality of any one type of therapeutic patch 14 or several different types of therapeutic patches 14.

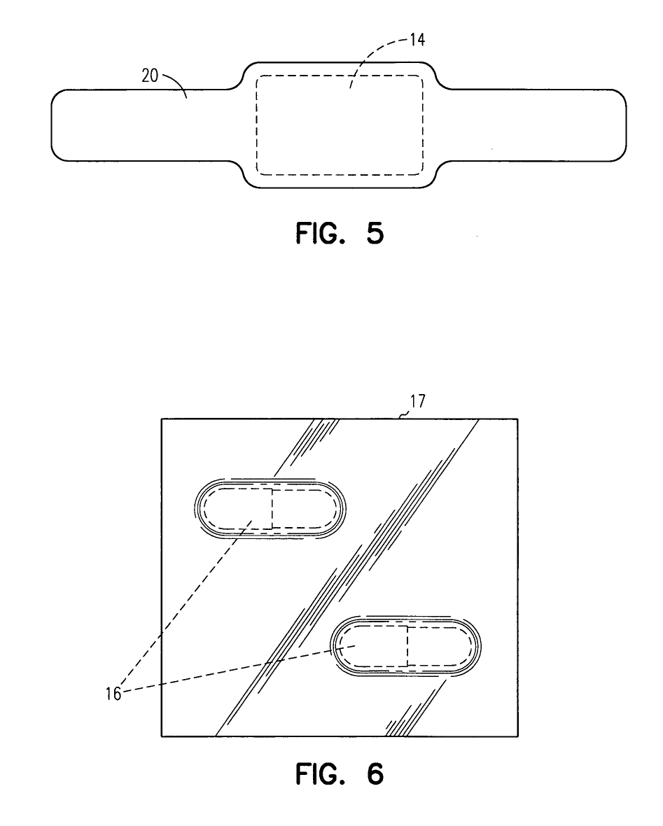

FIG. 4 shows one of the example therapeutic patches 14 of FIG. 3 where the therapeutic patch 14 is partially removed from an air-tight enclosure 15. It should be noted that some, none or all of the therapeutic patches 14 may be sealed inside some type of enclosure 15 before they are held by the container 12 (see e.g., FIG. 3). The need to seal a therapeutic patch 14 inside an air-tight enclosure will depend in part on the type of therapeutic patch 14.

As an example, therapeutic patch 14 may be a heat patch that generates heat via an exothermic reaction. Heat patches typically include a heating composition that must be exposed to air to begin the exothermic reaction. The heating composition within such heat patches may be exposed to air by removing the heat patch from an air-tight enclosure.

Referring again to FIGS. 1 and 2, the system 10 may further include a flexible wrap 20 for supporting a portion 101 of a body 100 and securing one or more of the therapeutic patches 14 near the portion 101 of the body 100. As an example, the flexible wrap 20 may be secured relative to the body 100 at a location near the injured portion 101 of the body. The therapeutic patch 14 (or patches) may then be placed against the injured portion 101 of the body 100 and held in place by wrapping the flexible wrap 20 around the injured portion 101 of the body 100 until an end of the flexible wrap 20 is secured to an already-wrapped section of the flexible wrap 20.

The flexible wrap 20 may be formed from one or more of layers. In addition, the flexible wrap 20 may include infrared energy reflectors that reflect infrared energy emitted by the body back into the injured portion of the body. The number and type of layers will depend on the application where the flexible wrap 20 is used. As an example, some layers may be more elastic while other layers may be made of softer and/or more insulating materials.

FIG. 5 shows an example flexible wrap 20 where the therapeutic patch 14 forms a part of the flexible wrap 20. The proper size and shape of the flexible wrap 20 will depend on the application where the flexible wrap 20 is used. It should be noted that more than one size and type of flexible wrap 20 may be included in system 10.

In the sample system 10 illustrated in FIG. 1, a plurality of oral pain relievers 16 are held by the container 12. Although three oral pain relievers 16 are shown in FIG. 1, the number and type of oral pain relievers 16 may vary depending on the type of system. As an example, one oral pain reliever 16 may be one type of drug and another oral pain reliever 16 may another type of drug. The choice of oral pain reliever 16 to apply will depend on the type of therapy required by the injured portion 101 (among other factors).

It should be noted that any type of oral pain reliever 16 may used in system 10 or any of the systems described herein. Some example oral pain relievers 16 include acetaminophen, ibuprofen, aspirin, naproxen sodium, ketoprofen and COX-2 selective NSAID (among others). As used herein, oral pain reliever is meant to refer to any item that is ingested to apply therapy to an injured portion of a body.

FIG. 6 shows an example oral pain reliever 16 that may be sealed inside an enclosure 17. It should be noted that some, none or all of the oral pain relievers 16 may be sealed inside some type of enclosure before they are held by the container 12. As an example, the system 10 may include a plurality of enclosures such that each enclosure includes an individual supply of the oral pain reliever 16.

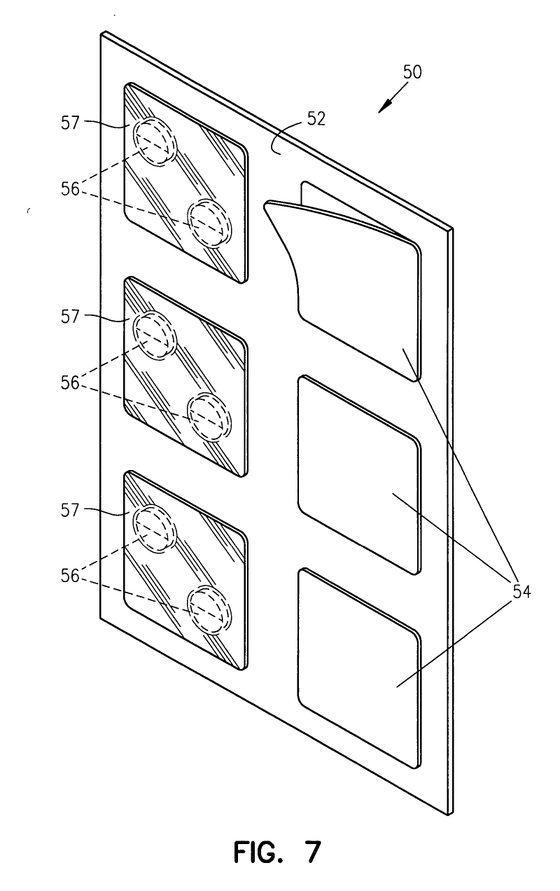

FIG. 7 illustrates another system 50 for providing therapy to a portion of a body. The system 50 includes a holder, such as board 52, and one or more therapeutic patches 54 that are releasably secured to the board 52. The therapeutic patches 54 apply therapy to the portion of the body when they are placed on, or near, the portion of the body. It should be noted that the system 50 may include any of the therapeutic patches described above.

The system 50 further includes one or more oral pain relievers 56 that are also releasably secured to the board 52. The oral pain relievers 56 apply therapy to the portion of the body when they are ingested within the body.

The therapeutic patches 54 and the oral pain relievers 56 may be applied to the board 52 using an adhesive, especially when the therapeutic patches 54 and the oral pain relievers 56 are within enclosures. In the example embodiment illustrated in FIG. 6, each enclosure 57 may include an individual supply of the oral pain reliever 56.

The therapeutic patches 54 may be peeled from the board 52 and applied near the portion of the body (see, e.g., the therapeutic patch 14 in FIG. 2). In addition, the enclosures 57 that store the oral pain relievers 56 may be peeled from the board 52 and then opened so that the oral pain reliever 56 may be ingested. The decision as to the number and type of therapeutic patches 54 and oral pain relievers 56 to use will depend on the type of therapy that is required by the injured portion of the body.

A method of providing therapy a portion 101 of a body 100 is described herein with reference to FIGS. 1-7. The method includes selecting a therapeutic patch (e.g., therapeutic patch 14) from a holder (e.g., container 12) that holds one or more therapeutic patches 14. The method further includes applying the patch 14 on, or near, the portion 101 of the body 100. It should be noted that applying the therapeutic patch 14 near the portion 101 of the body 100 may include wrapping the therapeutic patch 14 within a flexible wrap 20 which is spiraled around the injured portion 101 of the body 100 (see, e.g., FIG. 2).

The method further includes selecting an oral pain reliever 16 from the holder 12 and ingesting the oral pain reliever 16. In some sample forms of the method, ingesting the oral pain reliever 16 includes removing the oral pain reliever 16 from an enclosure 17 (see, e.g., FIG. 6).

In some sample forms of the method, selecting a therapeutic patch 14 from a holder 12 may include selecting one of a plurality of therapeutic patches 14 from the holder 12. In addition, selecting an oral pain reliever 16 from the holder 12 may include selecting one of a plurality of oral pain relievers 16 from the holder 12.

In some embodiments, selecting a therapeutic patch 14 from a holder 12 may include removing the therapeutic patch 14 from a container 12. In addition, selecting an oral pain reliever 16 from the holder 12 may include removing the oral pain reliever 16 from the container 12.

In other embodiments (see, e.g., FIG. 7), selecting a therapeutic patch 54 from a holder 52 may include releasing the therapeutic patch 54 from a board 52. In addition, selecting an oral pain reliever 56 from the holder 52 may include releasing the oral pain reliever 56 from the board 52.

There are some forms of the method where selecting a therapeutic patch 14 from a holder 12 may include selecting a heat patch and/or cooling patch (among other types of therapeutic patches) from the holder 12. In addition, selecting a therapeutic patch 14 from the holder 12 may include selecting a therapeutic patch 14 that includes an infrared energy reflector which reflects infrared energy emitted by the body back into the portion of the body.

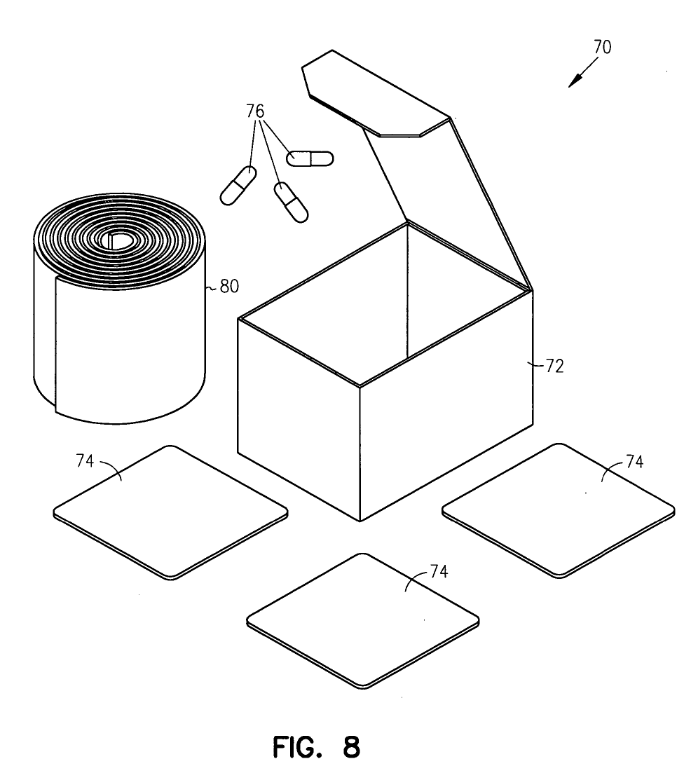

FIG. 8 shows a kit 70 of parts for providing therapy to a portion of a body. The kit includes one or more therapeutic patches 74 that are adapted to apply therapy to the portion of the body when the therapeutic patch 74 is placed near the portion of the body. The kit 70 also includes one or more oral pain relievers 76 that are adapted to relieve pain within the body when the oral pain reliever 76 is ingested into the body.

The kit 70 further includes a holder, such as container 72, which is adapted to hold the therapeutic patches 74 and the oral pain relievers 76. In other forms of the kit 70, the holder may be a board (see, e.g., board 52 in FIG. 7) such that the therapeutic patches 74 and the oral pain relievers 76 are releasably attached to the board.

The therapeutic patches 74 that are used in the kit 70 may be of any type. As examples, therapeutic patch 74 may be an exothermic heat patch that is adapted to heat to the body or an endothermic cooling patch that is adapted to cool the body. In addition, any of the therapeutic patches 74 that are used in the kit 70 may include an infrared energy reflector which reflects infrared energy emitted by the body back into the injured portion of the body. It should be noted that one or more of the therapeutic patches 74 which are used in the kit 70 may be sealed inside an enclosure (see, e.g., therapeutic patch 14 shown in FIG. 4).

The oral pain relievers 76 that are used in the kit 70 may be of any type. As an example, oral pain reliever 76 may include a non-steroidal anti-inflammatory drug. It should be noted that one or more of the oral pain relievers 76 which are used in the kit 70 may be sealed inside an enclosure such that each enclosure includes an individual supply of the oral pain reliever 76 (see, e.g., oral pain reliever 16 and enclosure 17 shown in FIG. 6).

The kit 70 may further include a flexible wrap 80 that is adapted to secure one or more of the therapeutic patches 74 in the kit 70 near the portion of the body. The proper size and shape of the flexible wrap 80 will depend on the application where the flexible wrap 80 is used (i.e., the size and shape of the injured portion on the body).

It should be noted that more than one size and type of flexible wrap 80 may be included in the kit 70. In addition, the therapeutic patch 74 may form part of the flexible wrap 80 (see, e.g., flexible wrap 20 in FIG. 5).

The operations discussed above with respect to the described methods may be performed in a different order from those described herein. It should be noted that attaching a therapeutic patch to a body includes attaching the therapeutic patch directly or indirectly to the body. In addition, FIGS. 1-8 are representational and are not necessarily drawn to scale. Certain proportions thereof may be exaggerated, while others may be minimized.

The systems and methods described herein may provide a user or therapist with the ability to readily apply a combination of therapies to an injured area of a body. The systems and methods may also reduce the amount of therapy-related items that would otherwise need to be inventoried in order to apply a combination of therapies to an injured portion of a body. In addition, the system and method may enhance the ability of an oral pain reliever to provide therapy to a particular injured area of a body.

While the invention has been described in detail with respect to the specific aspects thereof, it will be appreciated that those skilled in the art, upon attaining an understanding of the foregoing, may readily conceive of alterations to, variations of, and equivalents to these aspects which fall within the spirit and scope of the present invention, which should be assessed accordingly to that of the appended claims.

1. A system for providing therapy to a portion of a body, the system comprising:
a holder;
a therapeutic patch that is held by the holder, wherein the therapeutic patch applies a therapy to the portion of the body when the therapeutic patch is placed near the portion of the body; and
an oral pain reliever that is held by the holder.

2. The system of claim 1 further comprising at least one additional therapeutic patch held by the holder.

3. The system of claim 1 wherein the therapeutic patch is an exothermic heating patch.

4. The system of claim 1 wherein the therapeutic patch is an endothermic cooling patch.

5. The system of claim 1 wherein the therapeutic patch includes an electrical stimulating device.

6. The system of claim 1 wherein the holder is a container.

7. The system of claim 1 wherein the holder is a board.

8. The system of claim 1 further comprising a plurality of oral pain relievers that are held by the holder.

9. The system of claim 8 wherein the plurality of oral pain relievers are stored in a plurality of enclosures such that each enclosure includes an individual supply of the oral pain reliever.

10. The system of claim 1 wherein the therapeutic patch includes an infrared energy reflector that is capable of reflecting infrared energy emitted by the body back into the portion of the body.

11. The system of claim 1 wherein the oral pain reliever is a non-steroidal anti-inflammatory drug.

12. The system of claim 1 wherein the therapeutic patch is releasably attached to the holder.

13. The system of claim 1 further comprising a flexible warp that is capable of holding the therapeutic patch against the portion of the body.

14. The system of claim 13 wherein the therapeutic patch is part of the flexible wrap.

15. A kit of parts for providing therapy to a portion of a body, the kit comprising:
a therapeutic patch that is adapted to apply a therapy to the portion of the body when the therapeutic patch is placed near the portion of the body;
an oral pain reliever that is adapted to relieve pain from the body when the oral pain reliever is ingested within the body; and
a holder adapted to hold the therapeutic patch and the oral pain reliever.

16. The kit of claim 15 wherein the holder is a container.

17. The kit of claim 15 wherein the therapeutic patch is an exothermic patch adapted to heat to the body.

18. The kit of claim 15 wherein the therapeutic patch is an endothermic patch adapted to cool the body.

19. The kit of claim 15 wherein the therapeutic patch is within an enclosure.

20. The kit of claim 19 wherein the enclosure is a substantially air-tight enclosure.

21. The kit of claim 15 further comprising a flexible wrap that is adapted to secure the therapeutic patch near the portion of the body.

22. The kit of claim 15 wherein the therapeutic patch is adapted to be releasably secured to the holder and the oral pain reliever is adapted to be releasably secured to the holder.

23. The kit of claim 15 further comprising at least one additional therapeutic patch that is adapted to apply therapy to the portion of the body when the therapeutic patch is placed near the portion of the body.

24. The kit of claim 15 wherein the therapeutic patch includes an infrared energy reflector that is adapted to reflect infrared energy emitted by the body back into the portion of the body.

25. The kit of claim 15 wherein the oral pain reliever is a non-steroidal anti-inflammatory drug.

26. The kit of claim 15 further comprising a flexible warp that is adapted to hold the therapeutic patch against the portion of the body.

27. The kit of claim 26 wherein the therapeutic patch is part of the flexible wrap.

28. The kit of claim 15 further comprising a plurality of oral pain relievers that are adapted to relieve pain from the body when the oral pain relievers are ingested within the body.

29. The kit of claim 28 wherein the plurality of oral pain relievers are stored in a plurality of enclosures such that each enclosure includes an individual supply of the oral pain reliever.

30. A method of providing therapy to a portion of a body, the method comprising:
selecting a therapeutic patch from a holder;
applying the therapeutic patch near the portion of the body such that the therapeutic patch applies therapy to the portion of the body;
selecting an oral pain reliever from the holder; and
ingesting the oral pain reliever.

31. The method of claim 30 wherein applying the therapeutic patch near the portion of the body includes applying the therapeutic patch to the portion of the body.

32. The method of claim 30 wherein selecting a therapeutic patch from a holder includes selecting one of a plurality of therapeutic patches from the holder, and wherein selecting an oral pain reliever from a holder includes selecting one of a plurality of oral pain relievers from the holder.

33. The method of claim 30 wherein selecting a therapeutic patch from a holder includes removing the therapeutic patch from a container, and wherein selecting an oral pain reliever from the holder includes removing the oral pain reliever from the container.

34. The method of claim 30 wherein selecting a therapeutic patch from the holder includes releasing the therapeutic patch from a board, and wherein selecting an oral pain reliever from the holder includes releasing the oral pain reliever from the board.

35. The method of claim 30 wherein applying the therapeutic patch near the portion of the body includes wrapping a flexible wrap around the portion of the body to secure the therapeutic patch to the body.

36. The method of claim 30 wherein selecting a therapeutic patch from a holder includes selecting a heat patch from the holder.

37. The method of claim 30 wherein selecting a therapeutic patch from a holder includes selecting a cooling patch from the holder.

38. The method of claim 30 wherein selecting a therapeutic patch from the holder includes selecting a therapeutic patch that includes an infrared energy reflector which is capable of reflecting infrared energy emitted by the body back into the portion of the body.

39. The method of claim 30 wherein ingesting the oral pain reliever includes removing the oral pain reliever from an enclosure.
